(19) United States
(12) Patent Application Publication
(10) Pub. No.: US 2013/0303878 A1
(43) Pub. Date: Nov. 14, 2013

(54) SYSTEM AND METHOD TO ESTIMATE LOCATION AND ORIENTATION OF AN OBJECT

(71) Applicants: Erez Nevo, Natanya (IL); Abraham Roth, Kefar Hasidim (IL)

(72) Inventors: Erez Nevo, Natanya (IL); Abraham Roth, Kefar Hasidim (IL)

(73) Assignee: ENAV MEDICAL LTD., Yokneam (IL)

(21) Appl. No.: 13/981,043

(22) PCT Filed: Jan. 19, 2012
(86) PCT No.: PCT/IL12/50017

§ 371 Date: Jul. 22, 2013

Related U.S. Application Data
(60) Provisional application No. 61/434,512, filed on Jan. 20, 2011.
(60) Provisional application No. 61/440,873, filed on Feb. 9, 2011.

(51) Int. Cl. A61B 5/05

(52) U.S. Cl. CPC A61B 5/05
USPC 600/409

(57) ABSTRACT

A tracking system for estimating the position and orientation of an object inside a patient comprising electromagnets that generate magnetic fields used to navigate an object, including rotating and translating the object, are used to track the position of the object. Position tracking of the object is concurrent with navigating the object; or interleaved with navigating the object. Using the same electromagnets for navigation and tracking ensure coordinate system registration between the navigation system and the position tracking system. A tracking sensor attached to the object comprises at least a single coil generating signals in response to time varying tracking magnetic field generated by the electromagnets. Iterative algorithm is used to estimate position and orientation from sensor's signal. Linearly time varying current in the tracking electromagnets is produced by applying calculated voltage waveform to the electromagnet coils.

### FIELD OF THE INVENTION

The present invention relates to methodology and apparatus to determine the location and orientation of an object, for example a medical device, located inside or outside a body of a living subject. More specifically, the invention enables estimation of the location and orientation of various medical devices (e.g. catheters, surgery instruments, endoscopes, untethered capsules, etc.) by measuring electrical potentials induced by time-variable magnetic fields in a sensor having at least one sensing element as a coil. The invention further improves the generation of the magnetic fields required for the determining the location and orientation of the object.

### BACKGROUND

Remote Magnetic Navigation Systems (RMNS), employed by various companies (e.g. Stereotaxis, Inc.; Magnetecs, Inc.) is an emerging technology for use in catheterization, endoscopy, endoscopic capsule (“video pill”) and other minimally invasive procedures.

Catheters with magnetic tips can be steered within the patient, without the need for an electrophysiologist to maneuver the catheter manually. Unlike other robotic navigation techniques, the catheter is controlled by steering the distal tip with a magnetic field. The technology has been proven to reduce physician and patient exposure to radiation and procedure times, as well as enable more precise navigation of the vasculature with increased safety and efficacy [Pappone C and Santenelli V, Safety and efficacy of remote magnetic ablation for atrial fibrillation, J Am Coll Cardiol. 2008 Apr. 22; 51(16):1614-5]. Additionally, remote magnetic navigation increases catheter stability while reducing the temperature required to successfully perform an ablation [Davis D R, Tang A S et al., Remote magnetic navigation-assisted catheter ablation enhances catheter stability and ablation success with lower catheter temperatures, Pacing Clin Electrophysiol. 2008 July; 31(7):893-8].

Traditional catheter labs in hospitals rely on the manual placement and steering of catheters by a physician. In interventional cardiology, catheters are used to map the cardiovascular system and to correct arrhythmias and atrial fibrillation, among other heart related problems, through a variety of methods including ablation. The patient is placed under a fluoroscopic system, such as a C-arm, to give the electrophysiologist real-time feedback on the positioning of the catheter. In manual procedures, the physician must wear a lead apron due to radiation exposure, whereas with RMNS, the operator can conduct the procedure in a shielded room or at another location via a network connection. Then ablation catheters are used to burn scars in heart tissue to correct irregular rhythms. Apart from ablation, cardiologists use guide wires and catheters to place stents and other devices in the anatomy. Remote magnetic navigation operates by using large electromagnets placed in proximity to the patient, and alterations in the magnetic field produced by the electromagnets deflects the tips of catheters within the patient to the desired direction. The catheter itself is advanced by a remote controller like a joystick, instead of the physician's hands.

As of January 2009, 18,000 total clinical cases were performed by magnetic navigation according to Stereotaxis website, with a complication rate of less than 0.1%, representing a minute fraction of complications occurring with manual and other robotic navigation systems.

Another system has been introduced by Magnetecs Corporation. The robotic Catheter Guidance Control and Imaging (CGCI) system features an electromagnetic array consisting of eight stationary electromagnets in a spatial configuration that enables navigation of a magnetically-tipped catheter. CGCI system benefits include significant reduction of overall procedure time due to fast catheter maneuvering capability, real-time 3D and visual feedback for the physician, and the system's integrated real-time multi-media imaging combined with automated catheter control. The magnetic field within the CGCI structure eliminates the need for expensive added magnetic shielding in the operating room. Exposure to X-rays is reduced for the patient and eliminated for the physician. The CGCI system has two standard modes of control: Manual Magnetic mode and Automatic Magnetic control mode. The joystick-controlled Manual Magnetic mode provides a responsive way to direct the catheter tip about the chamber. The Automatic Magnetic mode gives the operator point-and-click targeting of map locations. In Automatic Magnetic mode, the CGCI logic routines plan a path to the targeted location, determine the optimal contact direction, and guide the catheter tip until it makes firm and continuous tissue contact. The CGCI system uses the static map geometry to plan a guidance path that will bring the catheter tip into contact with the moving tissue as it passes through the selected map location. (additional information may be found in Magentecs web site, http://magnetecs.com).

These magnetic navigation systems use auxiliary tracking system that track the object in order to enable the magnetic control of the object position and orientation. Thus the integration of Stereotaxis Niobe® Magnetic Navigation System with Biosense CARTO RMT System enables the closed-loop navigation of magnetically steered catheters. The CARTO RMT System tracks the location of the catheter in real time and shares this information with the Niobe System, allowing the physician to navigate the catheter from the control room. (Additional information may be found in http://www.biosensewebster.com/products/navigation/cartormt.aspx). The CARTO tracking system has several limitations—it uses solid sensors with three orthogonal coils, which cannot be used with lumen catheters or over very small guidewires; it uses electromagnetic coils to generate magnetic fields for tracking, which may interfere with the magnetic coils of the magnetic navigation system; since the magnetic navigation system and the tracking system use different magnetic fields for their tasks, there is a need to register the two coordinate systems (i.e. to define a coordinate transformation between the two systems).

The EndoScout tracking system for MRI (Robin Medical, Inc.) uses the gradient fields of the scanner as the reference fields for tracking, and thus has no electromagnetic interference with the scanner and there is no need to register the tracking system and the MRI scanner (Additional information may be found in www.robinmedical.com). Like the CARTO tracking sensor, the EndoScout tracking sensor is a solid sensor containing at least 3 orthogonal micro coils that cannot be used in guidewires and in lumen catheters.

As described in U.S. Pat. No. 6,516,213 to Nevo, the activations of gradient coils in MRI scanners provide the required data to estimate the location and orientation of a sensor that has at least 3 orthogonal coils. The estimation process is based on minimization of the difference between measured and predicted sensor signals. This can be done by various minimization methods, for example the minimization of the sum of squares of the differences between the measured and predicted signals (the least squares method). The measured signals in each of the sensor coils are linearly related to the time derivative of the magnetic flux through each coil respectively (Faraday Law of Induction). Thus the measured signals can be compared with reference signals that are calculated from the known distribution of the gradient fields in the scanner, the known pattern of gradient activation, and the known geometry of the tracking sensor.

As further described in patent application WO 2009/087601A2 to Roth and Nevo, additional gradient activations for tracking can be used with or without the gradient activations for imaging to improve the performance of the tracking system and to achieve more accurate tracking with faster update rate.

US application 20100280353A1, titled “method and apparatus to estimate location and orientation of objects during magnetic resonance imaging”, to Roth and Nevo, discloses a method for estimating location and orientation of medical device e.g. catheter, which involves processing instantaneous values of magnetic fields generated by activation of gradient coils based on command parameters for object tracking. Tracking based on the gradient fields of magnetic resonance imaging (MRI) scanners based on passive operation of the tracking system without any change of the scanner's hardware or mode of operation. To achieve better tracking performance, a technique to create a custom MRI pulse sequence is disclosed. Through this technique any standard pulse sequence of the scanner can be modified to include gradient activations specifically designated for tracking. These tracking gradient activations are added in a way that does not affect the image quality of the native sequence. The scan time may remain the same as with the native sequence or longer due to the additional gradient activations. The tracking system itself can use all the gradient activations (gradient activations for imaging and gradient activations for tracking) or eliminate some of the gradients and lock onto the specific gradient activations that are added to the custom pulse sequence.

US Patent Application 20110301497; titled “diagnostic and therapeutic magnetic propulsion capsule and method for using the same”; to Shachar, et. al.; discloses a guided medical propulsion capsule driven by strong electro-magnetic interaction between an external AC/DC magnetic gradient-lobe generator and a set of uniquely magnetized ferrous-conductive elements contained within the capsule. The capsule is navigated through the lumens and cavities of the human body wirelessly and without any physical contact for medical diagnostic, drug delivery, or other procedures with the magnetically guiding field generator external to the human body. The capsule is equipped with at least two sets of magnetic rings, disks and/or plates each possessing anisotropic magnetic properties. The external magnetic gradient fields provide the gradient forces and rotational torques on the internal conductive and magnetic elements needed to make the capsule move, tilt, and rotate in the body lumens and cavities according to the commands of an operator.

### SUMMARY OF THE INVENTION

There is a need for an integrated magnetic navigation and position tracking system that eliminates the need for system registration and thus increases the accuracy of the system.

There is also a need for systems and methods that are capable of providing positioning and orientation of a single coil so as to enable the integration of tracking sensors on the outer surface of guidewires and lumen catheters and to eliminate the need for coordinate system registration.

It is one object of the present invention to provide a method and apparatus for determining the instantaneous location and orientation of an object moving through a three-dimensional space, which method and apparatus have advantages in one or more of the above respects.

In the present application, a new tracking methodology and apparatus is disclosed. The disclosed method and system may be used to estimate the position and orientation of an object inside the operating field of RMNS.

In the present invention, the electromagnets that generate magnetic fields used to navigate an object, including rotating and translating the object, are used to track the position of the object. Position tracking the object may be done concurrent with navigating the object; or tracking the object can be interleaved with navigating the object. By using the same electromagnets to navigate and to track the object, there is no need for coordinate system registration between the navigation system and the position tracking system.

According to exemplary embodiments of the present invention, the sensor for measurement of an instantaneous magnetic field may comprise a coil assembly comprising one or more coils having axes of known orientations with respect to the sensor.

According to exemplary embodiments of the present invention, the sensor may comprise a plurality of sensor coils oriented in known orientations, and the data processing may comprise storing in memory reference magnetic field maps of each of the electromagnets in the host system, and simultaneously estimating the location and the orientation of the sensor by processing the measured instantaneous values of the magnetic fields generated by the tracking mode electromagnet activation together with the known reference magnetic field maps of the electromagnets and the known relative orientation of the sensor coils.

According to exemplary embodiments of the present invention, the sensor may comprise a coil assembly including one coil. In some embodiments, the single coil in the sensor may be planar, in other embodiments it may be a non-planar coil. In some embodiments, each sensor includes a pair of sensor coils, wherein a first sensor coil in the pair is parallel to, but laterally spaced from the second sensor coil of the pair. In some embodiments, each sensor includes two or more sensor coils, wherein all coils are positioned in known orientations and positions in the sensor. The sensor may be active sensor, such as a Hall-effect sensor, a passive sensor such as a coil sensor, or any other suitable sensor. In some embodiments, the object may be a medical instrument moving in the body of a person for medical diagnostic or treatment purposes. Examples include catheters, endoscopes, and capsules with wireless communication to a receiver outside the body.

According to yet additional exemplary embodiments of the present invention, the system may further comprise a triggering mechanism for triggering of the tracking mode electromagnets activation signal. In some embodiments, the tracking mode electromagnets activation signal is a bi-modal signal.

In some embodiments, the objects an ingestible capsule having very limited space for the tracking sensor and the signal conditioning and signal processing resources. One of the preferred activation waveforms is a triangular current signal. Specifically, a linear change of current may be preferred. It should be noted that a triangular waveform of the activation currents is only one preferred optional waveform. An advantage of the triangular activation waveform is the resulting flat plateau of the signal induced in the sensor coil. This plateau may reduce various artifacts and noise that are induced for example by the external magnets of the navigation system.

Accordingly, the current invention further provides an optional method of generating a linearly time-changing in magnetic field inside a coil-based magnetic field generator, by applying special waveform input voltage signals to a large coil. The voltage signals are calculated from the following parameters: the peaks (minimum and maximum) electric current in the coil; the time interval between these peaks, the resistance of the coil and the inductance of the coil.

According to an exemplary embodiments of the current invention a method for tracking a position of an object within a body the is provided, the method comprising: attaching a magnetic sensor to an object; positioning said object within a three-dimensional space within the a body; generating, using tracking electromagnets, at least five time-varying tracking magnetic fields within said three-dimensional space, said at least five magnetic fields comprising: at least two substantially spatially homogenous fields within a three-dimensional space; and at least three spatially gradient fields within a three-dimensional space; creating magnetic field map for each of said generated time-varying magnetic fields, said map charts the corresponding magnetic field vector at locations in said three-dimensional space; measuring the response of said magnetic sensor to said at least five time-varying magnetic fields; estimating the three-dimensional location, and at least two-dimensional orientation of said object within said three-dimensional space using said magnetic field maps and said measured response of said magnetic sensor to said at least five time varying magnetic fields.

In some embodiments estimating the location and orientation of said object comprises using iterative estimation algorithm.

In some embodiments the estimating a location and an orientation comprises minimizing the differences between said measured responses of said magnetic sensor expected response calculated using said magnetic field map.

In some embodiments the magnetic sensor comprises at least one magnetic detector.

In some embodiments the sensor comprises at least two magnetic detectors spatially displaced from each other.

In some embodiments the magnetic sensor comprises at least two magnetic detectors having different orientation with respect to each other.

In some embodiments estimating the location and orientation of said object comprises estimation the location of each of said at least two magnetic detectors.

In some embodiments the object is non-rigid such that said at least two magnetic detectors change at least one of: their relative orientation, and their relative position as said object changes its shape.

In some embodiments estimating the location and orientation of said non-rigid object further comprises estimation at least one parameter defining the change in shape of said non-rigid object.

In some embodiments the non-rigid object is a flexible catheter; having at least two magnetic detectors are located at known distances along said catheter; said at least one parameter defining the change in shape of said non-rigid object comprises flexing of said catheter.

In some embodiments at least one of said magnetic detectors is a Hall Effect probe.

In some embodiments at least one of said magnetic detectors is a coil.

In some embodiments measuring the response of said magnetic detector comprises measuring the voltage induced in at least one coil in response to said time-varying magnetic fields.

In some embodiments the method, further comprises: generating navigation magnetic fields by navigation electromagnets; and navigation of said object within said three-dimensional space by applying forces induced by said navigation magnetic fields on said object.

In some embodiments at least one of said navigation magnetic fields and at least one of said tracking magnetic field are generated by the same electromagnet.

In some embodiments the navigation magnetic fields and said tracking magnetic field are generated by the same set of electromagnets.

In some embodiments the electromagnets comprise at least one pair of Helmholtz coils.

In some embodiments the electromagnets comprise at least one pair of electromagnets having a ferromagnetic core.

In some embodiments the electromagnets comprise at least three pairs of opposing electromagnets external to said body, each of said three pairs of opposing electromagnets is configured to generate a set of magnetic fields within said three-dimensional space, wherein each of said sets is capable of generating a homogenous field and a gradient field.

In some embodiments the homogenous field is generated by activating a pair of opposing electromagnets with current flowing in the same direction for each electromagnet of said pair.

In some embodiments the gradient field is generated by activating a pair of opposing electromagnets with current flowing in an opposite direction for each electromagnet of said pair.

In some embodiments the method further comprising activating electromagnet of at least one of said pairs of opposing electromagnets with different currents.

In some embodiments the at least three pairs of electromagnets are positioned substantially orthogonally with respect to each of the other pairs.

In some embodiments the iterative optimization process is effected in real time to determine the instantaneous location and orientation of said object.

In some embodiments generating, said time-varying tracking magnetic fields comprises sequentially generating said time-varying magnetic field.

In some embodiments at least one of said sequentially generated said time-varying magnetic fields comprises of at least one time duration in which said field is linearly changing with time; and at least one of said magnetic detectors is a coil, such that the response of said magnetic detector to said time-varying magnetic field is substantially constant voltage during said time duration in which said field is linearly changing with time.

In some embodiments the object is a non-tethered object within a body cavity.

In some embodiments the object is an ingestible pill.

In some embodiments the time duration in which said field is linearly changing with time is overlap with a substantially constant field used for navigating said object.

In some embodiments the time-varying magnetic fields comprises a plurality of time durations in which said field is linearly changing with time.

In some embodiments the time-varying magnetic fields comprises a triangular waveform.

In some embodiments the linearly changing with time field is generated by activating at least one electromagnet with a linearly changing in time current, produced by a controlled voltage source, producing in said coil of said magnetic detector a linearly changing in time voltage during said time duration in which said field is linearly changing with time.

In some embodiments the controlled voltage source is configured to produce voltage waveform of Vin(t)={R·(i1−i0)/(t1−t0)}·t+{L·(i1−i0)/(t1−t0)+R·[i0]; for t0<t<t1, wherein: Vin(t) is the voltage time varying waveform; t is time variable; t0 and t1 are the beginning and the end respectively of said time duration in which said field is linearly changing with time; R is the total resistance of said electromagnet circuit loop; L is the total inductance of said electromagnet circuit loop; i0 is the current at time t0; and i1 is the current at time t1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically depicts a block illustration of a remote magnetic navigation system (RMNS), in accordance with embodiments of the present invention.

FIG. 2A schematically shows activation pattern of the RMNS electromagnets for tracking only.

FIG. 2B schematically depicts activation pattern of the RMNS electromagnets for both navigation and tracking.

FIG. 3A schematically depicts a sensor having a single coil.

FIG. 3B schematically depicts a sensor having a two sensor coils and.

FIG. 3C schematically depicts a flexible catheter having two sensor coils and.

FIG. 3D schematically depicts a flexible catheter having four sensor coils.

FIG. 3E schematically depicts a sensor having a single, non-planar sensor coil.

FIG. 3F schematically depicts a sensor having two non-parallel sensor coils.

FIG. 3G schematically depicts an exploded 3D view of a sensor having six sensor coils arranged in three pairs, wherein coils in each pair are substantially oriented along the same axis and displaced from each other along said axis, and the pair are oriented such that their axis are substantially orthogonal to each other.

FIG. 4A schematically depicts a possible configuration of electromagnets pairs in a tracking and navigation system.

FIG. 4B(i) schematically depicts front view of a possible configuration of six electromagnets pairs in a tracking and navigation system.

FIG. 4B(ii) schematically depicts side view of the configuration of six electromagnets pairs in a tracking and navigation system seen in FIG. 4b(i).

FIG. 5 schematically depicts the equivalent diagram of electromagnet activation circuitry.

FIG. 6A schematically depicts a graph showing an exemplary triangular electromagnet activation current as a function of time.

FIG. 6B schematically depicts a graph showing an exemplary triangular electromagnet activation voltage as a function of time needed to excite the current seen in FIG. 6A.

FIG. 7A schematically depicts a graph showing exemplary asymmetric electromagnet activation current as a function of time.

FIG. 7B schematically depicts a graph showing an exemplary asymmetric electromagnet activation voltage as a function of time needed to excite the current seen in FIG. 7A.

### DETAILED DESCRIPTION OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The terms “comprises”, “comprising”, “includes”, “including”, and “having” together with their conjugates mean “including but not limited to”.

The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In discussion of the various figures described herein below, like numbers refer to like parts. The drawings are generally not to scale. For clarity, non-essential elements were omitted from some of the drawing.

The present invention discloses apparatus, method and system to track the position of a sensor having at least one coil in a remote magnetic navigation system (RMNS) that has electromagnets that are activated to manipulate the position and/or orientation of an object inside the body of a living subject. The disclosed method, system and apparatus enable the estimation of the location and orientation of an object by using a magnetic sensor, for example a set of one or more miniature coils attached to the object.

An exemplary embodiment uses only one coil in the set. However more complex coil sets, for example a set of two or more coils, may improve the accuracy of the tracking. The following discloses a single coil sensor and a tracking sensor having more than one coil.

Complete (6 degrees of freedom) tracking of a position sensor attached to an object requires determination of orientation and of location of the sensor. The orientation of a single sensor coil may be determined by at least two substantially spatially homogenous, time-variable, magnetic fields which are substantially at different directions that induce potentials in the coil that depend on the relative orientation between the coil and each of the magnetic fields. In some embodiments the spatially homogenous, time-variable, magnetic fields are substantially mutually orthogonal to each other. The determination of orientation does not require prior knowledge of the location of the coil, since the magnetic fields are assumed to be spatially homogenous. Once the orientation of the coil is determined, its position may be determined by consecutive activation of gradient fields. In gradient fields, the field amplitude changes in space. When three gradient fields that change along the three axes of the coordinate system are activated, the induced voltages in the coil may be used to determine its position. Thus, the position and orientation of a single coil can be determined by consecutive application of 3 orthogonal gradient fields and at least two orthogonal homogenous fields. The axial rotation of a planar coil cannot be determined, since induction through the planar coil does not change with axial rotation of the coil, thus the tracking provides 5 Degrees Of Freedom (DOF) position of the sensor (3 location coordinates and 2 orientation coordinates).

If all 6 DOF of the sensor are needed, either a non-planar single coil can be used, or at least two coils can be used. In order to get the 6 unknown position parameters of a non-planar single coil, at least 6 field activations are needed, for example 3 gradient fields at substantially different directions and 3 homogenous field at substantially different directions. In some embodiments the gradient magnetic fields are substantially mutually orthogonal to each other. In some embodiments the spatially homogenous, time-variable, magnetic fields are substantially mutually orthogonal to each other. If a sensor equipped with two coils is used, at least 3 field activations are needed to determine the 6 unknown position parameters. However, more field activations may be used in order to provide more measurements than unknown parameters, which may be solved by methods for over-determined set of data like linear least squares (or other optimization algorithms known in the art).

The present invention provides a method of using the magnetic fields of the host RMNS (that are primarily used to navigate the object, i.e. to move it or to rotate it) for position tracking as well. Thus, there is no need to conduct coordinate system registration between the navigation and tracking systems), as in other tracking/navigation systems. In systems known in the art, separate transmitters having separate coordinate systems may need to be registered to the coordinate system of the host. In the present invention, the use of the same electromagnets to generate the fields of the host system and of the tracking system provides a significant improvement in accuracy, since a small error in the registration may result in a significant tracking error. Additionally, the present invention eliminates the need for additional field generators for position tracking, and eliminates possible electromagnetic interference between the tracking system and the navigating system. The elimination of the additional field generators may reduce system cost and/or complexity. Alternatively, separate electromagnets to generate the magnetic fields for tracking may be used and mechanically integrated with the magnets of the host RMNS to ensure fixed registration of the coordinate systems of the tracking system and the RMNS.

### System and Magnetic Field Configuration

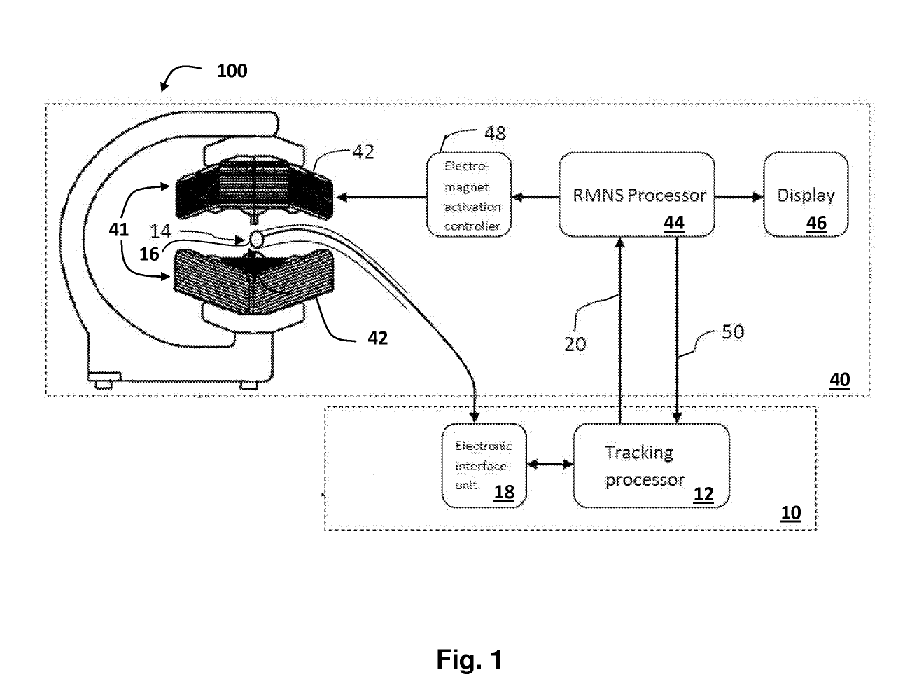

Reference is now made to FIG. 1, which is a schematic and block illustration of a remote magnetic navigation system (RMNS) 100, in accordance with embodiments of the present invention. RMNS 100 comprises an activation system 40, a tracking module 10, and an object 16. Object 16 may be a medical device, such as a catheter, a surgical instrument, an endoscope, an untethered capsule, or any other device which may be inserted into a body of a living subject. Activation system 40 includes an activation unit 41, an activation controller 48, an activation processor 44 and a display 46. Activation unit 41 comprises a set of electromagnets 42, positioned substantially opposed to one another. A body of a living subject (not seen in this figure for drawing clarity) may be placed within the set of electromagnets 42, and object 16 may be positioned on or in the body, and tracked by tracking module 10. Activation controller 48 controls electromagnets 42, and parameters used for activating electromagnets 42 may be varied. For example, the amplitudes and/or directions may be varied through activation controller 48. Activation processor 44 may receive data 20 from tracking module 10, may send data 50 to tracking module 10, and may send the received data to activation controller 48 for varying the activation parameters based on the received data. In addition or alternatively, activation processor 44 may send the received data to display 46 to enable control of navigation via visual feedback by the operator. Tracking module 10 comprises a tracking processor 12, a sensor 14 integrated into or attached to object 16, an electronic interface unit 18 between sensor 14 and tracking processor 12, and a tracking output 20. Data from sensor 14 is sent via electronic interface unit 18 to tracking processor 12. These data may then be sent 50 to activation processor 44 and subsequently processed and used for activating electromagnets 42. Alternatively, these data may be sent to activation processor 44 and then used to show a location and/or orientation of sensor 14 via display 46.

In an exemplary embodiments of the present invention, electromagnets 42 of activation system 40 may be operated in sequence to generate magnetic fields for magnetic navigation (navigation mode activation) and for tracking of object 16 (tracking mode activation), or activation patterns may be designed to enable navigation and position tracking.

If electromagnets 42 are positioned in opposing pairs, they can be operated in two different modes to produce two different types of field:

In the first mode, both electromagnets of each pair are activated by current flowing in the same direction, which results in a largely homogenous field between the pair of electromagnets; and

In a second mode, the two electromagnets are activated by current flowing in opposite directions, which results in a gradient field that has a gradual change of the magnetic field amplitude between the two electromagnets in each pair.

These two fields—the homogenous one and the gradient one—are considered to be a set of fields for each pair of opposing electromagnets. More general patterns of electromagnet activations can include activation of the two electromagnets by currents with different amplitudes and in the same or opposite direction resulting in various patterns of magnetic field distribution between the two electromagnets.

If a single, movable pair of electromagnets is used by the RMNS (For example as in the Niobe system of Stereotaxis, Inc.) to enable tracking of a position sensor, the pair may be positioned in different orientations with respect to the person being treated in order to generate at least three sets of magnetic fields where each set of fields has components that are mutually orthogonal to the other sets of fields.

If several pairs of opposing electromagnets are used by the RMNS (as in the CGCI system of Magnetecs, Inc.), the different pairs of electromagnets may be activated sequentially in order to generate at least three sets of magnetic fields where each set of fields have components that are mutually orthogonal to the other sets of fields. An alternative embodiment involves the use of separate electromagnets for navigation and for position tracking, where the different electromagnets are mechanically integrated to provide a fixed geometrical relation between the two sets of electromagnets and thus to ensure coordinate system registration between the two sets.

While position tracking of object 16 is needed continuously to enable navigation, activating the electromagnets for navigation may not be needed for long periods of times, or may use constant current during relatively long time (steady state activation) to navigate object 16. To accommodate the requirements of both navigation and continuous tracking, the electromagnets may be controlled to operate in two modes: navigation mode activation and tracking mode activation. Navigation mode activation may enable position tracking, for example if navigation is done by pulsating field activations (e.g. using Pulse Width Modulation (PWM)). However, if the electromagnet is activated for relatively long period of time in order to move the object from one location to another or to rotate it, rapid, bi-modal activations may be superimposed to enable position tracking.

In some embodiments of the invention, system 100 further comprises a navigation processor and user input (not seen in this figure). Optionally, the user may use the user input to direct object 16 to a desired location and/or orientation. Optionally a closed feedback loop in the navigation processor compares the actual location and/or orientation of object 16 as estimated by system 100 to the desired parameters entered by the user and issues corrective actions when needed by activating the electromagnets accordingly.

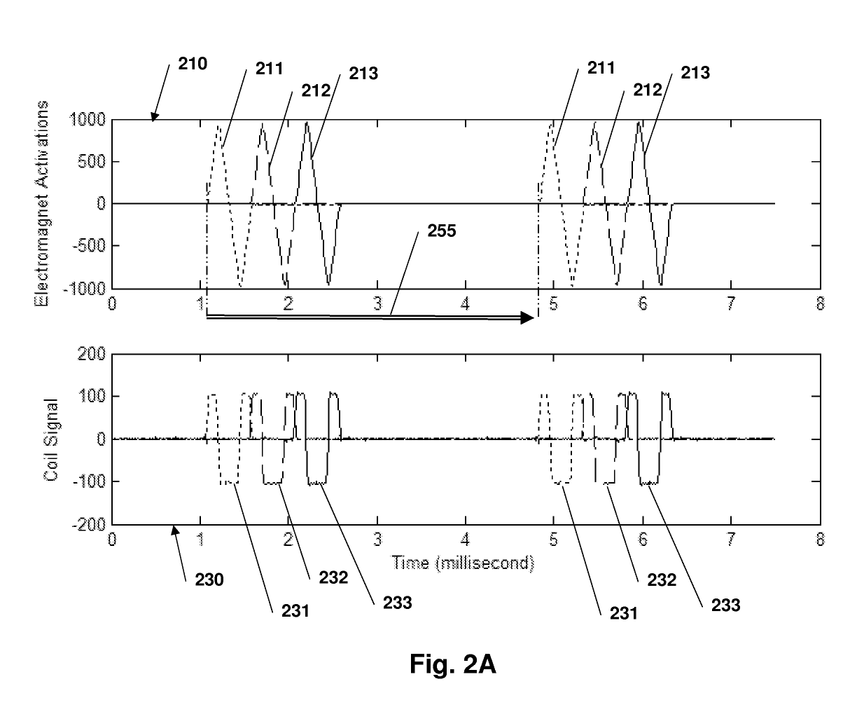

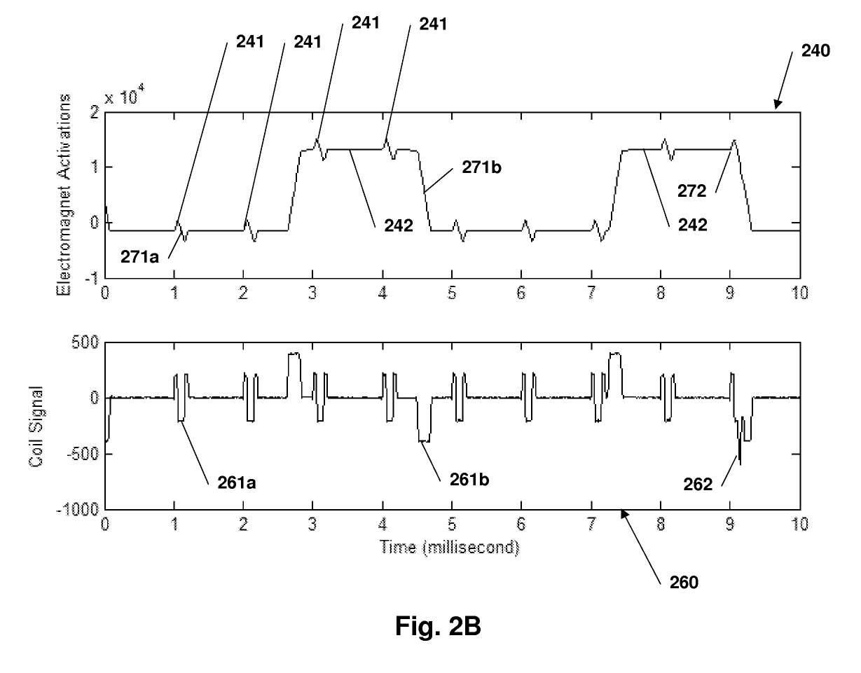

FIG. 2A shows activation pattern of the RMNS electromagnets for tracking only.

The top graph 210 schematically depicts the current in the electromagnets, and thus the generated magnetic flux generated by the electromagnets. The different types of lines: doted 211, dashed 212 and solid 213, depict the activation currents of three different electromagnets which are sequentially activated. It should be noted that activations of the different electromagnets need not be identical in amplitude, slope and repetition rate (the reciprocal of the repetition time 255). The activations of different electromagnets may not be adjacent. In the exemplary embodiments the activations of the different electromagnets are non-overlapping to avoid interference. Non-symmetric wavefunction may also be used.

The bottom graph 230 schematically depicts the voltage signal measured at the sensor coil. The different types of lines: doted 231, dashed 232 and solid 233, depict the signal at the sensor in response to the activation of the corresponding three different electromagnets which are sequentially activated.

It should be noted that signal generated at the sensor coil is proportional to the rate of change of the magnetic flux passing through the coil. Thus, the signal is substantially proportional to the time derivative of the electromagnet activation. The amplitude of the sensor's signal depends on the amplitude of the electromagnet activation, the coil size and number of turns, as well as other variables such the coil position and orientation relative to the electromagnets. Thus, in general the signal generated in response to activation of each electromagnet is different. It should be noted that the scales (time and amplitudes) of the graphs is for illustration purposes only.

FIG. 2B schematically depicts activation pattern of the RMNS electromagnets for both navigation and tracking.

If the object should not be moved, and the electromagnets are not activated for navigation, however, when navigation action is required, rapid bi-modal activations for tracking can be used. For drawing clarity, the activation (and sensor response) of only one electromagnet is depicted in this figure.

The top graph 240 schematically depicts the current in the electromagnet, and thus the generated magnetic flux generated by the electromagnet.

In the depicted example, a rapid repetition of low-amplitude tracking activations 241 (only four are marked) is superimposed on high-amplitude, less rapidly repeating navigation activations 242. Generally, while the navigational activation is used only when object 16 is to be moved, the tracking activation is used whenever the object is to be tracked.

The bottom graph 260 schematically depicts the voltage signal measured at the sensor coil.

The flat sections (for example 261a and 261b) in the coil's signal are caused by the constant slopes (271a and 271b) in the electromagnet activation. A complex coil's signal pattern (for example 262) is created whenever the slops of the navigation and tracking activations coincides 272

In the following description, the electromagnets that are used for tracking can be either the same electromagnets that are used for object navigation, or separate ones that are mechanically integrated with the electromagnets of the RMNS.

In the preferred embodiment, the position tracking sensor comprises at least one coil having many loops of conductive wire. Alternative magnetic sensors, for example Hall Effect sensor, may be used to monitor the generated magnetic field.

When a pair of opposing electromagnets 42 is activated, a time variable, spatial magnetic field B(t,x,y,z) is generated where x,y,z are coordinates along the three axes X, Y, Z of the RMNS coordinate system, and t is a time variable.

The magnetic fields that are generated by the electromagnets may be calculated from field maps that are generated by simulations, or are measured in different locations within the operating field of the RMNS by measuring the magnetic field amplitude and direction in a plurality of locations during activation of the electromagnets. These maps may be stored in various formats, for example as an array of three dependent variables (magnetic field components in the X,Y,Z directions of the magnetic field vector B) as function of three independent variables—the locations x,y,z. The electrical current in a specific electromagnet represents the time change of the magnetic field generated by this electromagnet, so the magnetic field as a function of time and location B(t,x,y,z) can be represented by multiplication of the magnet field map values by the current flow time-varying signal.

For proper operation, it is preferable that activation processor 44 and tracking module 10 would be synchronized. That is that the timing of each field activation are preferably known such that measurements of signals 231-233 may be performed at appropriate timing and may be interpreted correctly to yield the location and/or orientation of the object. In some embodiments, measurements are performed during the flat part 261a of the signal, corresponding to the constant linear change 271a in the tracking electromagnet activation. This synchronization may be achieved using the data exchange lines 20 and/or 50 seen in FIG. 1. Alternatively, signals from the sensor coil (or coils) may be monitored and timing information extracted from these signals. For example synchronization may be achieved using a Phase Lock Loop (PLL) circuit as known in the art. In these embodiments, tracking module 10 may be independent of activation system 40, and in these cases tracking module 10 may further comprise a display and other user's input and output devices.

In some embodiments, for example wherein the object is an ingestible capsule synchronization may be done wirelessly, for example via RF link between tracking processor 12, which is preferably external to the patient and the electronic interface unit 18 which is (at least to some degree) internal to the ingestible capsule. In an ingestible capsule, synchronization derived from sensor's signal requires only a transmitter in the capsule to transmit the detected information instead of bi-directional communication for both synchronization and measured data.

It should be noted that the triangular waveform of the activation currents is only one preferred optional waveform. It should be noted that other waveforms such as (but not limited to) triangular, sinusoidal, etc. may be used. An advantage of the triangular activation waveform is the resulting flat plateau 261 of the signal induced in the sensor coil. This plateau may reduce various artifacts and noise that are induced for example by the external magnets of the navigation system.

In some embodiments, for example wherein the object is an ingestible capsule having very limited space for the coil and signal conditioning and signal processing resources, reducing noise and interference may be more important.

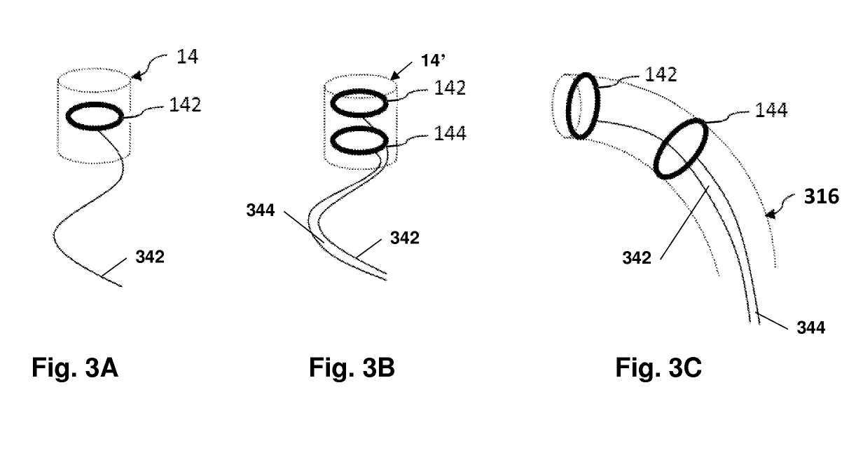

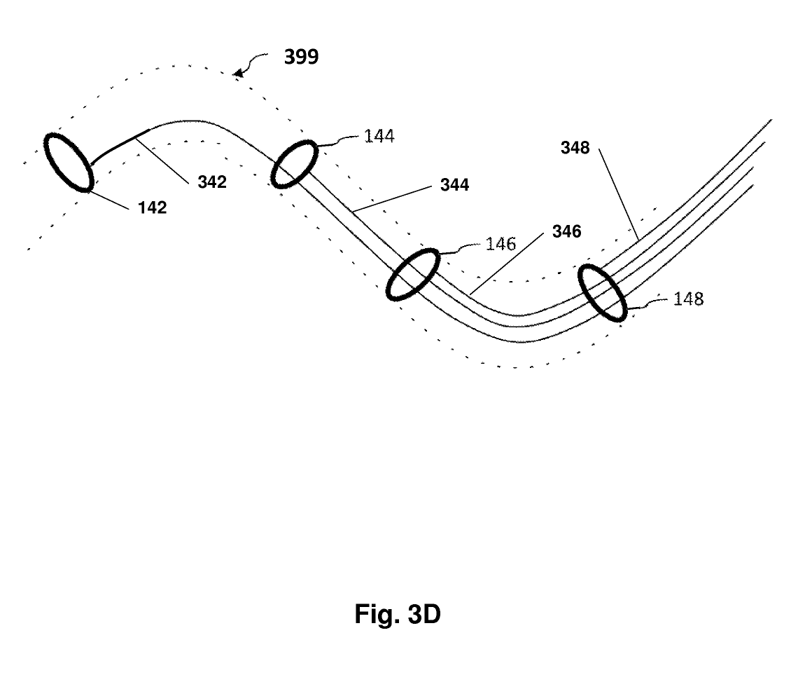

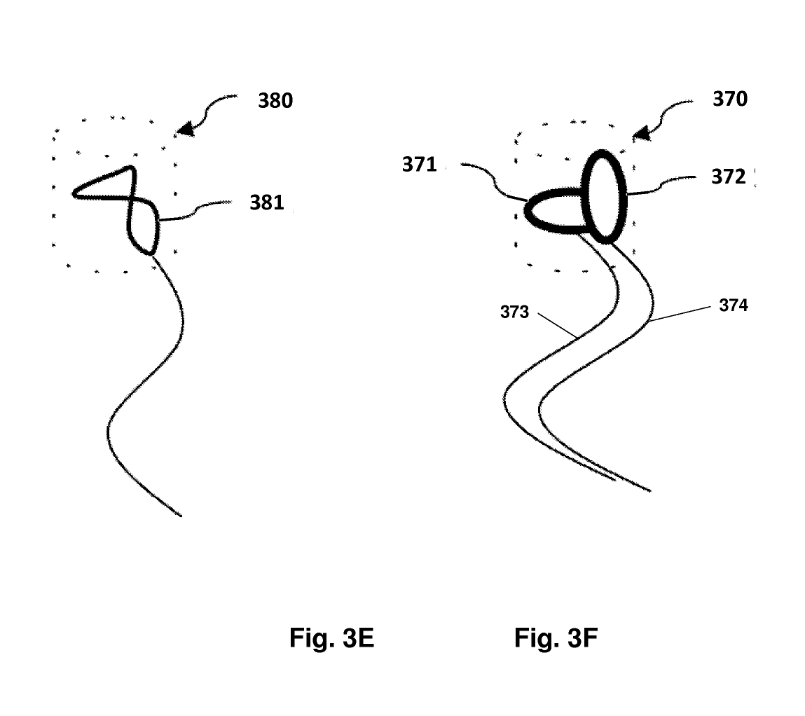

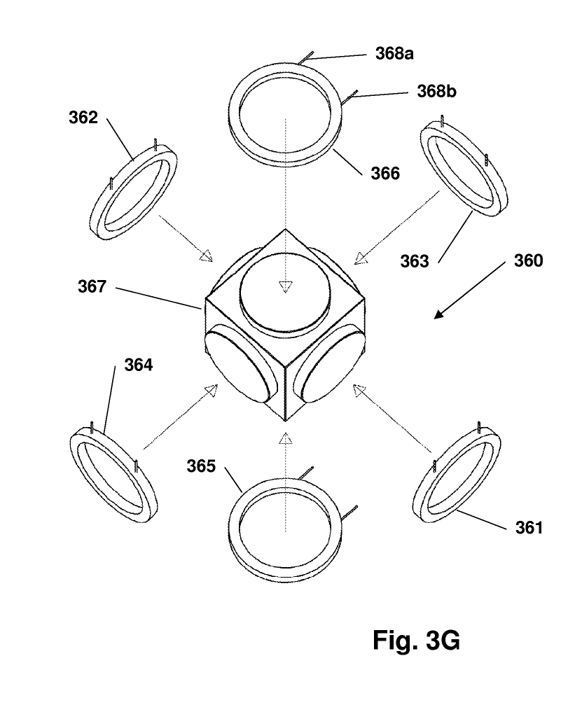

FIG. 3A schematically depicts a sensor 14 having a single coil 142.

In one embodiment, as shown in FIG. 3A, sensor 14 comprises one sensing coil 142. The time varying magnetic field B(t,x,y,z) induces electric potential in sensing coil 142, and the magnitude of the induced potential V is related to the time-derivative of the magnetic flux Θ through the coil, as given by the Faraday Law of Induction:

V=−dΘ/dt (1)

The magnetic flux through sensing coil 142 is determined by the magnetic field amplitude at the location of the coil, denoted by B(t,x,y,z), the coil area (A), and the angle between the magnetic field vector direction and the orientation of the coil represented by a unit direction vector n vertical to the plane of the coil:

Θ(t,x,y,z)=B(t,x,y,z)nA (2)

where denotes the vectorial dot product. A typical sensing coil 142 has multiple wire turns to increase its inductivity, so the area A represents the total induction area of the coil.
By using equations 1-2, one can predict the electrical potential Vp that is induced in the coil by the time variable magnetic field:

Vp=−d[B(t,x,y,z)nA]/dt (3)

The magnetic field B(t,x,y,z) is generated by repetitive activation of the external electromagnets of the RMNS. Various patterns of activations can be used. For example, for a single coil sensor at least 5 different fields are preferably activated in order to estimate the 5 unknown location parameters (3 coordinates and direction vector). Additional activations can be used to improved the tracking accuracy by solving an over-determined estimation problem (i.e. the number of data points is larger than the number of unknowns).

For example, in RMNS system as disclosed in US patent application US 2011/0301497 the 6 different electromagnets can be activated consecutively to generate 6 different magnetic fields for tracking. In this case, the B(t,x,y,z) field can be represented by these 6 magnetic fields:

B(t,x,y,z)=B1(t,x,y,z)+B2(t,x,y,z)+B3(t,x,y,z)+B4(t,x,y,z)+B5(t,x,y,z)+B6(t,x,y,z) (4)

where B1, B2, . . . B6 are the fields generated by activation of each electromagnet when all other electromagnets are not activated.

An alternative approach is to activate the electromagnets in three pairs where a pair has two parallel electromagnets. This enables the generation of fields with high level of spatial change in amplitude (gradient fields) or fields with low level of spatial change in amplitude (homogenous fields, typically termed Helmholtz fields). These specific fields are of interest since they are used by the RMNS—the gradient fields are used to translate the object while the homogenous fields are used to rotate the object. In this case the B(t,x,y,z) fields can be represented by:

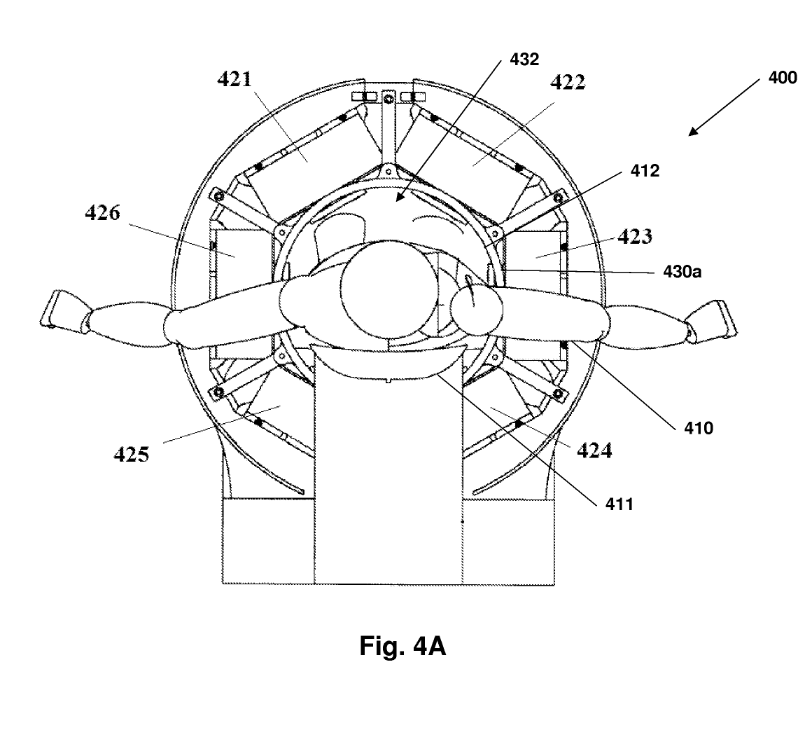

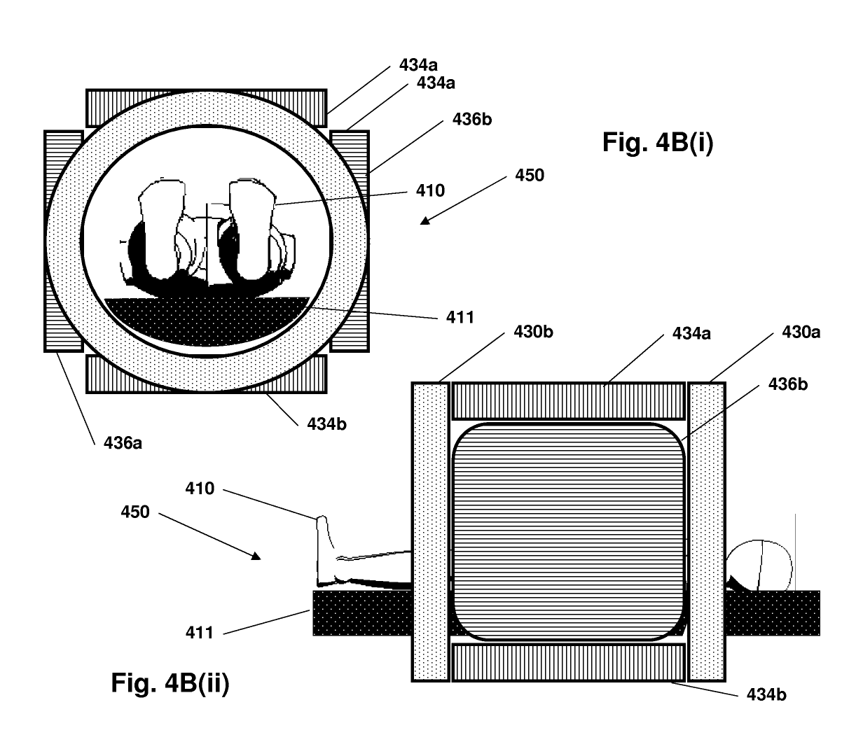

B(t,x,y,z)=G1(t,x,y,z)+G2(t,x,y,z)+G3(t,x,y,z)+H1(t,x,y,z)+H2(t,x,y,z)+H3(t,x,y,z) (5)

where G1, G2, and G3 are the gradient fields generated by electromagnets pairs {421,424} {422,425} {423,426} (as seen in FIG. 4). and H1, H2, H3 are the homogenous fields generated by the same pairs.

FIG. 4A schematically depicts a possible configuration of electromagnets pairs in a tracking and navigation system 400.

Patient 410 is positioned on a stretcher 411 such that its body is within the bore 412 surrounded by electromagnets 421-426 which are arranged in three opposing pairs: {421,424}; {422,425}; and {423,426}.

Optionally, a pair of coils 430a and 430b (only the front coil 430a a can be seen in this figure) are positioned with their axis parallel to the bore 432 through which patient 410 is positioned on opposite sides of said bore, to provide magnetic field in the direction along the length of the patient.

Similar configuration may be seen in FIG. 4B

FIG. 4B(i) schematically depicts front view of a possible configuration of six electromagnets pairs in a tracking and navigation system 450.

FIG. 4B(ii) schematically depicts side view of the configuration of six electromagnets pairs in a tracking and navigation system 450 seen in FIG. 4b(i).

The six coils configuration of FIGS. 4B(i) and 4B(ii) comprises:

A longitudinal pair of coils comprising a front coil 430a and a back coil 430b;

A vertical pair of coils comprising a top coil 434a and a bottom coil 434b; and

A horizontal pair of coils comprising a right coil 436a and a left coil 436b; and

It is apparent that a man skilled in the art of magnetism may design other electromagnet configurations within the general scope of the current invention.

The Iterative Estimation of Location and Orientation

Iterative estimation of the location and orientation is based on minimization of the differences between the measured induced potentials and the potentials that are predicted to be induced by the operation of the time-variable magnetic fields. In order to predict the induced potential in sensor coil 142, the location and orientation of sensor 14 should be given. Thus, when the estimation process is started, an initial guess of the location and orientation of the sensor is given by three position variables (e.g. the sensor coordinates xₒ, yₒ, zₒ in a Cartesian coordinate system of the RMNS) and a unit vector nₒ that represents the sensor direction (normal to the coil area). Once the location and orientation of the coil in the coordinate system of the RMNS is determined, the predicted electrical potential on the coil can be calculated by equation 3 and compared with the measured electrical potential (in this presentation of a single coil sensor we define the sensor coordinates as the center of the coil):

Vp(t)=−d[B(t,xₒ,yₒ,zₒ)nₒA]/dt

The actual electrical potential induced in the coil may be amplified by the signal conditioning system, so appropriate calibration is applied on the measured signals to yield the level of the measured electrical potential Vm.

The differences between the measured and predicted electrical potentials on sensor coil 142 during the activation of the electromagnets are used to calculate a Cost Function (CF) for the minimization algorithm of the iterative solution (for example, but not limited to, the sum of squares of the differences between the measured and predicted values):

CF=Θ(Vmᵢ−Vpᵢ)² (7)

where the sub-index i indicates a time region where a specific magnetic field i is generated by the electromagnets of the RMNS and measurement Vmᵢ is collected.

New values for the sensor location and orientation may be calculated by using standard minimization procedures that search for the location and orientation that minimize the cost function (for example, but not limited to the Levenberg-Marquardt search algorithm).

In the description above the cost function is based on at least five different measurements (one sensor coil during the activation of at least five different magnetic fields) and can be used to estimate the five unknown location and orientation parameters. The small number of measurements compared with the number of unknowns may result in inaccurate tracking due to noise in the measurements. To improve the performance, additional measurements can be acquired by using a second coil that is positioned in a known relative orientation and a known distance from the first coil (for example, two parallel coils 142, 144 in the sensor, as seen in FIG. 3B).

### Multi-Coil Sensor Configurations

FIG. 3B schematically depicts a sensor 14′ having a two sensor coils 142 and 144.

Coils 142 and 144 are at fixed known relative position to each other and the signals of each coil may be separately measured for example by connecting coils 142 and 144 to the electronics interface unit 18 with two separate cables 342 and 344 respectively. It should be noted that coils 142 and 144 need not be identical, and their orientation may not be parallel to each other.

Since the relative position of the second coil is known in reference to the first coil, the number of unknowns remains the same (five) while the number of measurements increases to 10. This redundancy in measurements generally increases the accuracy of the estimation.

FIG. 3C schematically depicts a flexible catheter 316 having two sensor coils 142 and 144.

Coils 142 and 144 are at fixed known distance from each other and the signals of each coil may be separately measured for example by connection coils 142 and 144 to the electronics interface unit 18 with two separate cables 342 and 344 respectively.

An alternative configuration allows constrained motion between the two coils, for example the two coils 142, 144 are placed on a flexible portion of the catheter 316, such that the distance between the two coils along the catheter is fixed and known, but the orientation of the second coil relative to the first coil may change due to catheter bending. In this case, the orientation of the second coil can be considered as an additional variable to be determined by the tracking algorithm, thus adding the two orientation parameters to the list of unknowns (total of seven unknown), while the position of the second coil can be calculated from the position of the first coil, the orientations of the two coils, and a geometrical model that represents the banding pattern of the catheter.

Compare to a single coil configuration of FIG. 3A, the number of unknowns is seven while the number of measurements increases to 10. This redundancy in measurements generally increases the accuracy of the estimation.

FIG. 3D schematically depicts a flexible catheter 399 having four sensor coils 142, 144, 146, and 148.

Coils 142 144, 146, and 148 are at fixed known distance from each other and the signals of each coil may be separately measured for example by connecting the coils 142 144, 146, and 148 to the electronics interface unit 18 with separate cables 342, 344, 346 and 348 respectively.

It should be noted that the number of coils may be smaller or larger than four, that the coils need not be identical, and their orientation relative to the long axis of the catheter 399 and relative to each other may be different.

Additional coils 146, 148 may be added along the object 399 as shown in FIG. 3D to provide information on the shape of the object during operation. This may be of particular use in cardiac catheter ablation where the shape of the ablation is controlled to achieve the required therapeutic effect. It is also noted that adding sensor coils having some spatial known relationship to each other (constrains) increases the number of measurements more than the increase in additional degrees of freedom. Specifically, for a rigid object the number of unknown remains the same. For a semi-rigid or flexible catheter, the number of degrees of freedom may increase by only two or three for each additional coil (defined by the unknown orientation due to catheter deflection, but position and in some cases rotation are constrained by the mechanical structure of the catheter), while the number of measurements increases by five (or by the number of different activations used in the measurements if different than five).

FIG. 3E schematically depicts a sensor 380 having a single, non-planar and non-symmetric sensor coil 381.

This special shape of the coil enables tracking of rotation around the axis of the coil, which is not possible with simple planar coil. It should be noted that non-planar sensor coil 381 may have an arbitrary 3D shape and the depicted shape is for illustration only.

FIG. 3F schematically depicts a sensor 370 having two non-parallel sensor coils 381 and 382.

Coils 381 and 382 are at fixed known relative position to each other and the signals of each coil may be separately measured for example by connecting coils 381 and 382 to the electronics interface unit 18 with two separate cables 383 and 384 respectively. It should be noted that coils 381 and 382 need not be identical, and their orientation may not be at right angle to each other.

FIG. 3G schematically depicts an exploded 3D view of a sensor 360 having six sensor coils 361-366 arranged in three pairs: {361,362}; {363,364}; and {365, 366}, wherein coils in each pair are substantially oriented along the same axis and displaced from each other along said axis, and the pair are oriented such that their axis are substantially orthogonal to each other.

Sensor 360 comprises a body 367 supporting coils 361-366 at fixed known relative position to each other. Preferably the signals of each coil may be separately measured for example by connection each coil separately to the electronics interface unit 18 with separate leads (for drawing clarity, only leads 368a and 368b of coil 366 are marked in this figure). It should be noted that the coils need not be identical, some may be missing, and they may be connected in series or in parallel to reduce the number of cabled leading to the electronics interface unit.

If all 6 location and orientation parameters are required, a sensor with a single non-planar coil (as seen in FIG. 3E) may be used.

Alternatively, if all 6 location and orientation parameters are required, a sensor with at least two coils (371 and 372) in different orientations (as seen in FIG. 3F) may be used.

For the single non-planar coil at least 6 different activations of the magnetic fields are needed to enable the estimation of the 6 position unknowns. When a sensor with two coils is used, at least 3 different activations of the magnetic fields are needed, but better tracking performance can be achieved with more activations or with more coils (for example as seen in FIG. 3G).

When the iterative process achieves the correct location and orientation of the sensor, the differences between the measured and predicted potentials will become small and the cost function will reach its minimal level (it may not reach the zero level due to various inaccuracies—for example noise in the measured signals, inaccuracy in the magnetic field maps, inaccuracy in the calibration of the signal conditioning system, limited numerical precision of the computation, etc.). The iterative process is stopped when the cost function achieves a small enough value, or when the level of reduction of the cost function becomes too small, or after a preset number of iterations, and the final set of coordinates is transferred from the tracking system to the RMNS system as an updated location of the tracking sensor.

### Improved Electromagnet Activation

As was noted in FIGS. 2A and 2B, one of the preferred activation waveforms is a triangular current signal such as 211-213, 241. Specifically, a linear change of current 271a may be preferred. accordingly, the current invention further provides an optional method of generating a linearly time-changing in magnetic field inside a coil-based magnetic field generator, by applying special waveform input voltage signals to a large field producing coil.

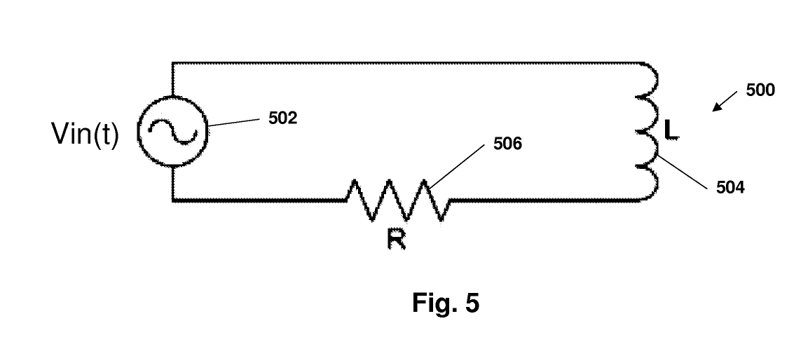

FIG. 5 schematically depicts the equivalent diagram 500 of electromagnet activation circuitry, wherein: Vin(t) 502 is the time varying voltage source; Inductance L 504 represents the total inductance of the electromagnet coil (or coils); and the resistance R 506 represents the total resistance in the loop such as the resistances of the power source, the electromagnet coil, the cables between source and coils and optimally intentional resistor inserted into the circuit (for example for suppressing transients and oscillations).

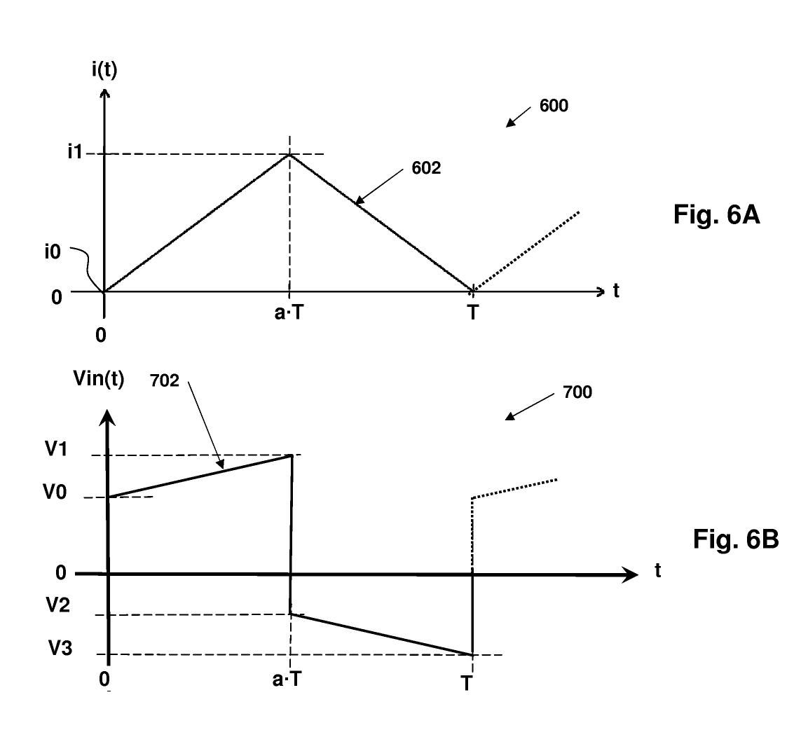

FIG. 6A schematically depicts a graph 600 showing an exemplary triangular electromagnet activation current i(t) 602 as a function of time. The minimum current in this example is i0=0 at times t=0 and t=T, and reaches its maximum i1 at time a·T wherein “a” is the asymmetry factor 0<a<1, such that a symmetric waveform is when a=0.5. The current waveform may optionally be repeated as depicted schematically by the dotted line.

In the following figures the time current and voltage scales are in arbitrary units.

An advantage of the triangular activation waveform is the resulting flat plateau 261 of the signal induced in the sensor coil. This plateau may reduce various artifacts and noise that are induced for example by the external magnets of the navigation system. It should be noted that the triangular waveform of the activation currents is only one preferred optional waveform.

In an RL circuit such as seen in FIG. 5, the current in the field producing coil 504 does not directly follows the voltage at source 502. Controlled current sources are often more complex and expensive than controlled voltage source and may require current feedback loops. In contrast, controlled voltage sources are easily commercially available and may be programmed to produce simple or complex desired output voltage waveforms. Programmable voltage sources are available which are capable of producing simple and complex voltage waveforms.

Accordingly, the current invention further provides an optional method of generating a linearly time-changing in magnetic field inside a coil-based magnetic field generator, by applying special waveform input voltage signals to a large coil. The voltage signals are calculated from the following parameters:
the current peaks (or, rather, the minimum current and maximum current between which the electric current signal changes linearly) in the coil i0 and i1 respectively;
the time interval between these peaks T;
The asymmetry factor a
the resistance of the field producing coil R; and
the inductance of the field producing coil L.

FIG. 6B schematically depicts a graph 700 showing an exemplary triangular electromagnet activation voltage Vin(t) 702 as a function of time needed to excite the current i(t) 602 in electromagnet 504. The voltage waveform may optionally be repeated as depicted schematically by the dotted line.

According to the exemplary embodiment, the voltage waveform 702 needed to create the current waveform 602 is given by the following function:
Starting at voltage V0 at time t=0 and linearly increasing to V1 at time t=a·T;
Rapidly decreasing the voltage at t=a·T to V2; and
Linearly decreasing the voltage from V2 at time=a·T to V3 at time t=T;
   Wherein:

V0=(i1·L)/(a·T)

V1=i1·R+(i1·L)/(a·T)

V2=i1·R−(i1·L)/((1−a)·T)

V3=−(i1·L)/((1−a)·T)

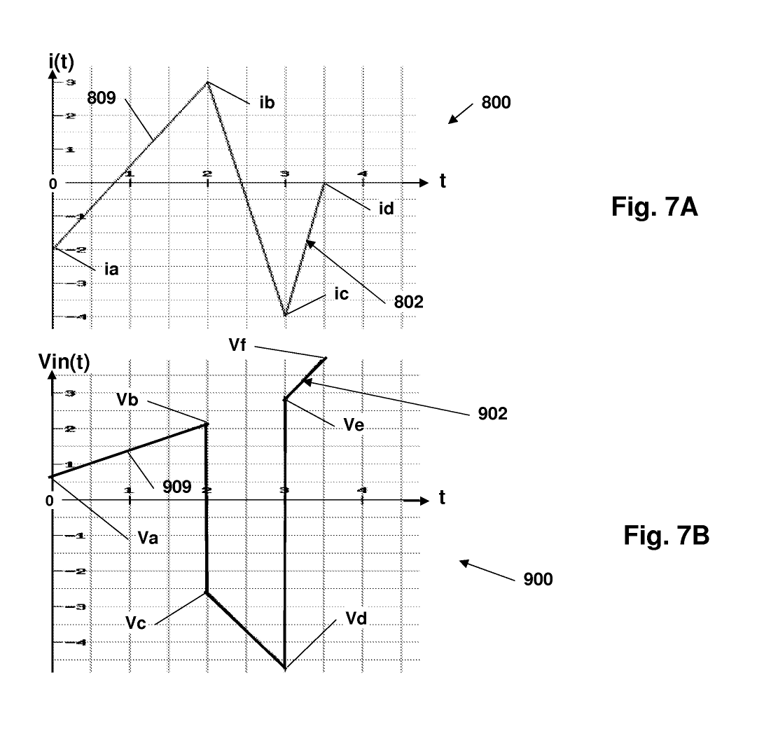

FIG. 7A schematically depicts a graph 800 showing an exemplary asymmetric electromagnet activation current i(t) 802 as a function of time.

In this exemplary waveform:

the initial current ia=−2 at t=0;

the max current ib=3 at t=2;

the minimum current ic=−4 at t=3; and

the final current id=0 at t=3.5

FIG. 7B schematically depicts a graph 900 showing the corresponding activation voltage Vin(t) 902 as a function of time needed to excite the current i(t) 802 in electromagnet 504.

According to the exemplary embodiment, L=0.5 [H], R=0.3 [Ohm] and the voltage waveform 902 needed to create the current waveform 802 is given by the following function

Starting at voltage Va=0.65 at time t=0 and linearly increasing to Vb=2.15 at time t=2;

Rapidly decreasing the voltage at t=2 to Vc=−2.6;

Linearly decreasing the voltage from Vc=−2.6 at time t=2 to Vd=−4.7 at time t=3;

Rapidly increasing the voltage at t=3 to Ve=2.8; and

Linearly increasing the voltage from Ve=2.8 at time t=3 to Vf=4 at time t=3.5

These and other input voltage waveform may be derived from the following equations:

The supply voltage V(in is given by:

Vin(t)=V_{L}(t)+V_{R}(t),

wherein V_{L}(t), the voltage on the coil is given by V_{L}(t)=L·di/dt; and
V_{R}(t)=i(t)·R; where di/dt is the time derivative of the current i(t).
The magnetic field produced in the field producing coil is proportional to the current and is given by:

B(t)=i(t)·L/(N·A);

wherein N is the number of turns in the coil and A is the area of the coil.
In each of the linear section of the current waveform, the currant i(t) may be expressed by the linear form:

i(t)=K0·t+K1;

where K0 is the slop and K1 is the value of the current at t=0;
thus the voltage needed may be expressed by:

Vin(t)=L·K0+R·(K0·t+K1)=(R·K0)·t+(L·K0+R·K1)

It is clear to see that the source voltage Vin(t) also follows a linear form.
Thus, in a general way, for a linear section in the current waveform i(t) starting at time t=t0 at current i(t)=i0 and ending at time t=t1 at current i(t)=i1, i(t) may be expressed as:

i(t)=K0·t+K1; wherein

K0=(i1−i0)/(t1−t0); and

K1=i0−K0·t0=i0−t0·(i1−i0)/(t1−t0).

And thus the voltage may be expressed by the linear form:

$$\begin{matrix}\begin{matrix}Vin ( t ) = ( R \cdot KO ) \cdot t + ( L \cdot KO + R \cdot K 1 ) \\ = \{ R \cdot ( i 1 + i 0 ) / ( t 1 - t 0 ) \} \cdot t + \\ \{ L \cdot ( i 1 - i 0 ) / ( t 1 - t 0 ) + R \cdot [ i 0 ] \} ;\end{matrix} & \\ for t 0 < t < t 1 & \end{matrix}$$

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for tracking a position of an object within a body the method comprising:
attaching a magnetic sensor to an object;
positioning said object within a three-dimensional space within the a body;
generating, using tracking electromagnets, at least five time-varying tracking magnetic fields within said three-dimensional space, said at least five magnetic fields comprising:
at least two substantially spatially homogenous fields within a three-dimensional space; and
at least three spatially gradient fields within a three-dimensional space;
creating magnetic field map for each of said generated time-varying magnetic fields, said map charts the corresponding magnetic field vector at locations in said three-dimensional space;
measuring the response of said magnetic sensor to said at least five time-varying magnetic fields;
estimating the three-dimensional location, and at least two-dimensional orientation of said object within said three-dimensional space using said magnetic field maps and said measured response of said magnetic sensor to said at least five time varying magnetic fields.

2-3. (canceled)

4. The method of claim 1, wherein said magnetic sensor comprises at least one magnetic detector

5. The method of claim 4, wherein said magnetic sensor comprises at least two magnetic detectors spatially displaced from each other.

6. The method of claim 4, wherein said magnetic sensor comprises at least two magnetic detectors having different orientation with respect to each other.

7. (canceled)

8. The method of claim 5, wherein said object is non-rigid such that said at least two magnetic detectors change at least one of:
their relative orientation, and
their relative position,
as said object changes its shape.

9. The method of claim 8, wherein said estimating the location and orientation of said non-rigid object further comprises estimation at least one parameter defining the change in shape of said non-rigid object.

10-11. (canceled)

12. The method of claim 1, wherein at least one of said magnetic detectors is a coil and wherein measuring the response of said magnetic detector comprises measuring the voltage induced in at least one coil in response to said time-varying magnetic fields.

13. (canceled)

14. The method of claim 1, further comprising:
generating navigation magnetic fields by navigation electromagnets; and
navigation of said object within said three-dimensional space by applying forces induced by said navigation magnetic fields on said object.

15. (canceled)

16. The method of claim 8, wherein said navigation magnetic fields and said tracking magnetic fields are generated by the same set of electromagnets.

17-18. (canceled)

19. The method of claim 1, wherein said electromagnets comprise at least three pairs of opposing electromagnets external to said body, each of said three pairs of opposing electromagnets is configured to generate a set of magnetic fields within said three-dimensional space, wherein each of said sets is capable of generating a homogenous field and a gradient field.

20-22. (canceled)

23. The method of claim 19, wherein said at least three pairs of electromagnets are positioned substantially orthogonally with respect to each of the other pairs.

24. (canceled)

25. The method of claim 1 wherein said generating, said time-varying tracking magnetic fields comprises sequentially generating said time-varying magnetic fields.

26. The method of claim 25 wherein:
at least one of said sequentially generated said time-varying magnetic fields comprises of at least one time duration in which said field is linearly changing with time; and
at least one of said magnetic detectors is a coil, such that the response of said magnetic detector to said time-varying magnetic field is substantially constant voltage during said time duration in which said field is linearly changing with time.

27. The method of claim 26 wherein said object is a non-tethered object within a body cavity.

28. The method of claim 17 wherein said object is an ingestible pill.

29. The method of claim 26 wherein said time duration in which said field is linearly changing with time is overlap with a substantially constant field used for navigating said object.

30. The method of claim 26 wherein said time-varying magnetic fields comprises a plurality of time durations in which said field is linearly changing with time.

31. The method of claim 30 wherein said time-varying magnetic fields is generated by activating at least one electromagnet with a non-linearly changing in time current, produced by a controlled voltage source, during said time duration in which said field is non-linearly changing with time.

32. The method of claim 26 wherein said linearly changing with time field is generated by activating at least one electromagnet with a linearly changing in time current, produced by a controlled voltage source, producing in said coil of said magnetic detector a substantially constant voltage during said time duration in which said field is linearly changing with time.

33. The method of claim 32 wherein said controlled voltage source is configured to produce voltage waveform of Vin(t)={R·(i1−i0)/(t1−t0)}·t+{L·(i1−i0)/(t1−t0)+R·[i0]}; for t0<t<t1
wherein:
Vin(t) is the voltage time varying waveform;
t is time variable;
t0 and t1 are the beginning and the end respectively of said time duration in which said field is linearly changing with time;
R is the total resistance of said electromagnet circuit loop;
L is the total inductance of said electromagnet circuit loop;
i0 is the current at time t0; and
i1 is the current at time t1.
